(19) United States
(12) Patent Application Publication
(10) Pub. No.: US 2016/0157785 A1
(43) Pub. Date: Jun. 9, 2016

(54) DEVICES, SYSTEMS, AND METHODS FOR DETECTING ANOMALOUS CARDIAC WAVEFORMS AND MAKING PHYSIOLOGIC MEASUREMENT CALCULATIONS

(71) Applicant: VOLCANO CORPORATION, San Diego, CA (US)

(72) Inventors: Fergus Merritt, Escondido, CA (US); Andrew Tochterman, Carlsbad, CA (US); John Unser, Temecula, CA (US); David Anderson, Temecula, CA (US)

(21) Appl. No.: 14/961,601

(22) Filed: Dec. 7, 2015

Related U.S. Application Data
(60) Provisional application No. 62/089,073, filed on Dec. 8, 2014.

(51) Int. Cl. A61B 5/00; A61B 5/0215; A61B 5/02

(52) U.S. Cl. CPC A61B 5/7246; A61B 5/02007; A61B 5/0215; A61B 5/7278; A61B 5/7203

(57) ABSTRACT

Devices, systems, and methods automatically detecting anomalous waveforms and eliminating these waveforms from physiologic measurements are disclosed. For example, in some instances a method includes collecting a pressure data from an intravascular device positioned within the vessel of the patient, the pressure data including a pressure waveform for each cardiac cycle of the patient; comparing the pressure waveform for each cardiac cycle of the patient to a reference pressure waveform to identify an anomalous pressure waveform; and calculating a pressure ratio utilizing the pressure data from the intravascular device, wherein data from the anomalous pressure waveform is excluded from the calculation.

### CROSS REFERENCE TO RELATED APPLICATIONS

The present application claims priority to and the benefit of the U.S. Provisional Patent Application No. 62/089,073, filed Dec. 8, 2014, which is hereby incorporated by reference in its entirety.

### TECHNICAL FIELD

The present disclosure relates generally to the assessment of vessels and, in particular, the assessment of the severity of a blockage or other restriction to the flow of fluid through a vessel. Aspects of the present disclosure are particularly suited for evaluation of biological vessels in some instances. For example, some particular embodiments of the present disclosure are specifically configured for the evaluation of a stenosis of a human blood vessel by detecting and excluding (or otherwise processing) data associated with anomalous cardiac waveforms from physiologic measurements/calculations.

### BACKGROUND

A number of techniques are currently used to assess the health of the blood vessels of a patient and in particular the severity of a stenosis in a blood vessel. Many of these techniques require analysis of the cardiac waveforms of a patient and may include physiologic measurements such as fractional flow reserve (FFR), instantaneous wave-free ratio (iFR), pressure ratios such as proximal pressure over distal pressure (Pa/Pd), coronary flow reserve (CFR), or electrocardiogram readings (ECG). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Physiologic measurements/calculations such as those described above are useful in diagnosing patients, but must have a high degree of reliability to be clinically useful. Anomalous heartbeat cycles can cause significant errors and/or deviations in the resulting physiologic measurements/calculations. In making physiologic measurements such as FFR, iFR, and/or CFR, underlying sets of cardiac waveforms (e.g., pressure waveforms, flow waveforms, ECG waveforms, etc.) are relied upon. The measurements may require averaging specific aspects of the waveform set(s) to make a diagnosis and corresponding treatment. Since the diagnosis and subsequent treatment options may depend on subtle variations between waveforms, anomalous waveforms that are included in the analysis may exaggerate some features of the waveform and reduce the resulting accuracy of the physiologic measurements/calculations. Current methods of filtering out anomalous waveforms are overly simplistic (e.g., using ECG readings of r-waves to filter out waveforms based on the total length of the cardiac cycle) and these methods lack the precision necessary to exclude many anomalous waveforms that can have a significant impact on physiologic measurements/calculations.

Accordingly, there remains a need for improved systems and methods for detecting and excluding (or otherwise processing) data associated with anomalous cardiac waveforms from physiologic measurements/calculations.

### SUMMARY

Embodiments of the present include a method of evaluating a vessel of a patient that includes collecting a pressure data from an intravascular device positioned within the vessel of the patient, the pressure data including a pressure waveform for each cardiac cycle of the patient; comparing the pressure waveform for each cardiac cycle of the patient to a reference pressure waveform to identify an anomalous pressure waveform; and calculating a pressure ratio utilizing the pressure data from the intravascular device, wherein data from the anomalous pressure waveform is excluded from the calculation. The reference pressure waveform can be based on a previously recorded set of pressure waveforms of the patient. Also, the reference pressure waveform can be fixed or variable during a procedure. For example, in some instances, the reference pressure waveform is variable and based on n previous pressure waveforms obtained during the procedure. The reference pressure waveform can also be selected from a database of available pressure waveforms. Comparing the pressure waveform for each cardiac cycle of the patient to the reference pressure waveform can include comparing a total cycle length, comparing a mean pressure, comparing a range between a maximum pressure and a minimum pressure, comparing a slope of a portion of the waveform, and/or other features of the pressure waveforms.

Devices and systems for implementing such methods are also disclosed.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic perspective view of a vessel having a stenosis according to an embodiment of the present disclosure.

FIG. 2 is a diagrammatic, partial cross-sectional perspective view of a portion of the vessel of FIG. 1 taken along section line 2-2 of FIG. 1.

FIG. 3 is a diagrammatic, partial cross-sectional perspective view of the vessel of FIGS. 1 and 2 with instruments positioned therein according to an embodiment of the present disclosure.

FIG. 4 is a diagrammatic, schematic view of a system according to an embodiment of the present disclosure.

FIG. 5 is a graphical representation of a reference waveform and measurements of associated physical features according to an embodiment of the present disclosure.

FIG. 6 is a graphical representation of a reference waveform and measurements of associated physical features according to another embodiment of the present disclosure.

FIG. 7 is a graphical representation of comparison of a reference waveform and a patient waveform and measurements of associated physical features.

FIG. 8 is a flow chart describing a method of automatically detecting and excluding anomalous waveforms from physiologic measurements according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

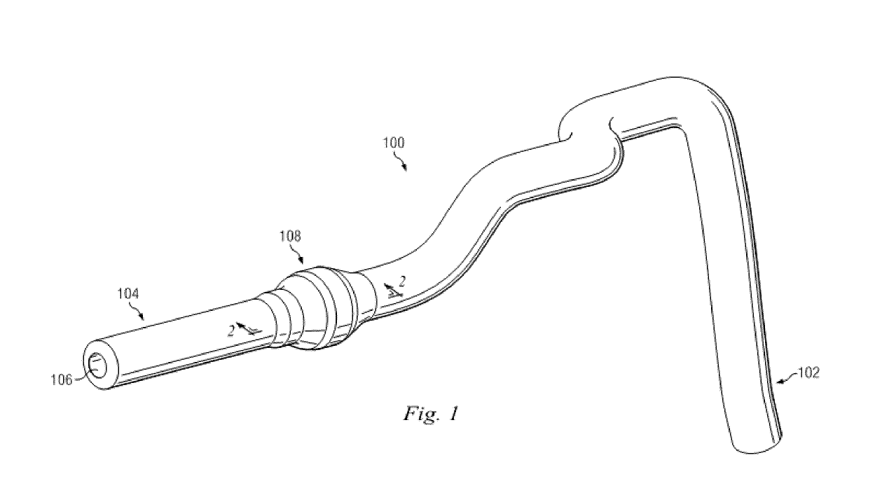

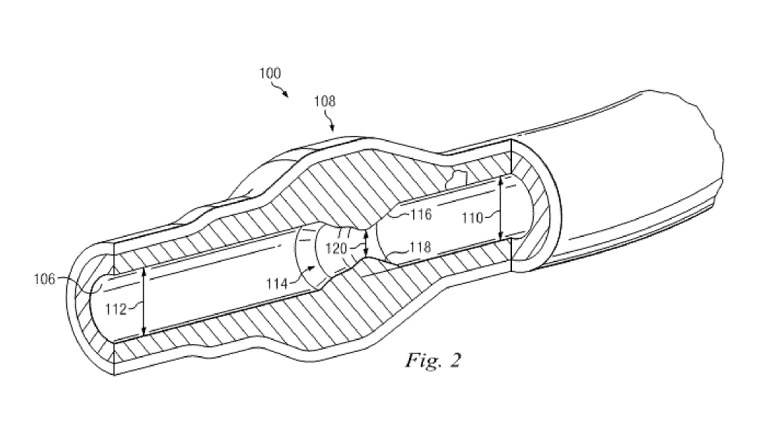

The present disclosure relates to physiologic measurements of a vessel such as FFR, iFR, pressure ratios such as proximal pressure over distal pressure (Pa/Pd), CFR or ECG readings. Referring to FIGS. 1 and 2, shown therein is a vessel 100 having a stenosis according to an embodiment of the present disclosure. In that regard, FIG. 1 is a diagrammatic perspective view of the vessel 100, while FIG. 2 is a partial cross-sectional perspective view of a portion of the vessel 100 taken along section line 2-2 of FIG. 1. Referring more specifically to FIG. 1, the vessel 100 includes a proximal portion 102 and a distal portion 104. A lumen 106 extends along the length of the vessel 100 between the proximal portion 102 and the distal portion 104. In that regard, the lumen 106 is configured to allow the flow of fluid through the vessel. In some instances, the vessel 100 is a systemic blood vessel. In some particular instances, the vessel 100 is a coronary artery. In such instances, the lumen 106 is configured to facilitate the flow of blood through the vessel 100.

As shown, the vessel 100 includes a stenosis 108 between the proximal portion 102 and the distal portion 104. Stenosis 108 is generally representative of any blockage or other structural arrangement that results in a restriction to the flow of fluid through the lumen 106 of the vessel 100. Embodiments of the present disclosure are suitable for use in a wide variety of vascular applications, including without limitation coronary, peripheral (including but not limited to lower limb, carotid, and neurovascular), renal, and/or venous. Where the vessel 100 is a blood vessel, the stenosis 108 may be a result of plaque buildup, including without limitation plaque components such as fibrous, fibro-lipidic (fibro fatty), necrotic core, calcified (dense calcium), blood, fresh thrombus, and mature thrombus. Generally, the composition of the stenosis will depend on the type of vessel being evaluated. In that regard, it is understood that the concepts of the present disclosure are applicable to virtually any type of blockage or other narrowing of a vessel that results in decreased fluid flow.

Referring more particularly to FIG. 2, the lumen 106 of the vessel 100 has a diameter 110 proximal of the stenosis 108 and a diameter 112 distal of the stenosis. In some instances, the diameters 110 and 112 are substantially equal to one another. In that regard, the diameters 110 and 112 are intended to represent healthy portions, or at least healthier portions, of the lumen 106 in comparison to stenosis 108. Accordingly, these healthier portions of the lumen 106 are illustrated as having a substantially constant cylindrical profile and, as a result, the height or width of the lumen has been referred to as a diameter. However, it is understood that in many instances these portions of the lumen 106 will also have plaque buildup, a non-symmetric profile, and/or other irregularities, but to a lesser extent than stenosis 108 and, therefore, will not have a cylindrical profile. In such instances, the diameters 110 and 112 are understood to be representative of a relative size or cross-sectional area of the lumen and do not imply a circular cross-sectional profile.

As shown in FIG. 2, stenosis 108 includes plaque buildup 114 that narrows the lumen 106 of the vessel 100. In some instances, the plaque buildup 114 does not have a uniform or symmetrical profile, making angiographic evaluation of such a stenosis unreliable. In the illustrated embodiment, the plaque buildup 114 includes an upper portion 116 and an opposing lower portion 118. In that regard, the lower portion 118 has an increased thickness relative to the upper portion 116 that results in a non-symmetrical and non-uniform profile relative to the portions of the lumen proximal and distal of the stenosis 108. As shown, the plaque buildup 114 decreases the available space for fluid to flow through the lumen 106. In particular, the cross-sectional area of the lumen 106 is decreased by the plaque buildup 114. At the narrowest point between the upper and lower portions 116, 118 the lumen 106 has a height 120, which is representative of a reduced size or cross-sectional area relative to the diameters 110 and 112 proximal and distal of the stenosis 108. Note that the stenosis 108, including plaque buildup 114 is exemplary in nature and should be considered limiting in any way. In that regard, it is understood that the stenosis 108 has other shapes and/or compositions that limit the flow of fluid through the lumen 106 in other instances. While the vessel 100 is illustrated in FIGS. 1 and 2 as having a single stenosis 108 and the description of the embodiments below is primarily made in the context of a single stenosis, it is nevertheless understood that the devices, systems, and methods described herein have similar application for a vessel having multiple stenosis regions.

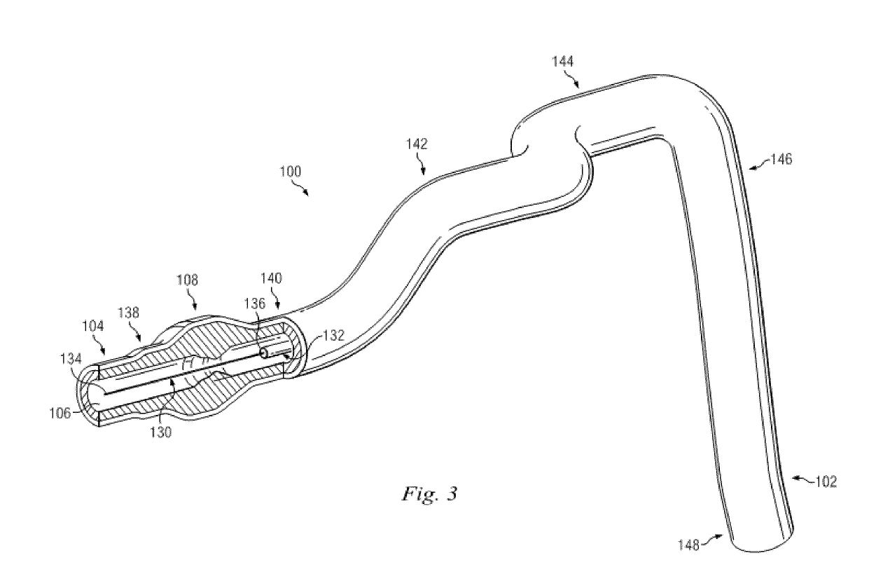

Referring now to FIG. 3, the vessel 100 is shown with instruments 130 and 132 positioned therein according to an embodiment of the present disclosure. In general, instruments 130 and 132 may be any form of device, instrument, or probe sized and shaped to be positioned within a vessel. In the illustrated embodiment, instrument 130 is generally representative of a guide wire, while instrument 132 is generally representative of a catheter. In that regard, instrument 130 extends through a central lumen of instrument 132. However, in other embodiments, the instruments 130 and 132 take other forms. In that regard, the instruments 130 and 132 are of similar form in some embodiments. For example, in some instances, both instruments 130 and 132 are guide wires. In other instances, both instruments 130 and 132 are catheters. On the other hand, the instruments 130 and 132 are of different form in some embodiments, such as the illustrated embodiment, where one of the instruments is a catheter and the other is a guide wire. Further, in some instances, the instruments 130 and 132 are disposed coaxial with one another, as shown in the illustrated embodiment of FIG. 3. In other instances, one of the instruments extends through an off-center lumen of the other instrument. In yet other instances, the instruments 130 and 132 extend side-by-side. In some particular embodiments, at least one of the instruments is as a rapid-exchange device, such as a rapid-exchange catheter. In such embodiments, the other instrument is a buddy wire or other device configured to facilitate the introduction and removal of the rapid-exchange device. Further still, in other instances, instead of two separate instruments 130 and 132 a single instrument is utilized. In that regard, the single instrument incorporates aspects of the functionalities (e.g., data acquisition) of both instruments 130 and 132 in some embodiments.

Instrument 130 is configured to obtain diagnostic information about the vessel 100. In that regard, the instrument 130 includes one or more sensors, transducers, and/or other monitoring elements configured to obtain the diagnostic information about the vessel. The diagnostic information includes one or more of pressure, flow (velocity), images (including images obtained using ultrasound (e.g., IVUS), OCT, thermal, and/or other imaging techniques), temperature, and/or combinations thereof. The one or more sensors, transducers, and/or other monitoring elements are positioned adjacent a distal portion of the instrument 130 in some instances. In that regard, the one or more sensors, transducers, and/or other monitoring elements are positioned less than 30 cm, less than 10 cm, less than 5 cm, less than 3 cm, less than 2 cm, and/or less than 1 cm from a distal tip 134 of the instrument 130 in some instances. In some instances, at least one of the one or more sensors, transducers, and/or other monitoring elements is positioned at the distal tip of the instrument 130.

The instrument 130 includes at least one element configured to monitor pressure within the vessel 100. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element are implemented as a solid-state component manufactured using semiconductor and/or other suitable manufacturing techniques. Examples of commercially available guide wire products that include suitable pressure monitoring elements include, without limitation, the PrimeWire PRESTIGE® pressure guide wire, the PrimeWire® pressure guide wire, and the ComboWire® XT pressure and flow guide wire, each available from Volcano Corporation, as well as the PressureWire™ Certus guide wire and the PressureWire™ Aeris guide wire, each available from St. Jude Medical, Inc. Generally, the instrument 130 is sized such that it can be positioned through the stenosis 108 without significantly impacting fluid flow across the stenosis, which would impact the distal pressure reading. Accordingly, in some instances the instrument 130 has an outer diameter of 0.035″, 0.018″, 0.014″ or less.

Instrument 132 is also configured to obtain diagnostic information about the vessel 100. In some instances, instrument 132 is configured to obtain the same diagnostic information as instrument 130. In other instances, instrument 132 is configured to obtain different diagnostic information than instrument 130, which may include additional diagnostic information, less diagnostic information, and/or alternative diagnostic information. The diagnostic information obtained by instrument 132 includes one or more of pressure, flow (velocity), images (including images obtained using ultrasound (e.g., IVUS), OCT, thermal, and/or other imaging techniques), temperature, and/or combinations thereof. Instrument 132 includes one or more sensors, transducers, and/or other monitoring elements configured to obtain this diagnostic information. In that regard, the one or more sensors, transducers, and/or other monitoring elements are positioned adjacent a distal portion of the instrument 132 in some instances. In that regard, the one or more sensors, transducers, and/or other monitoring elements are positioned less than 30 cm, less than 10 cm, less than 5 cm, less than 3 cm, less than 2 cm, and/or less than 1 cm from a distal tip 136 of the instrument 132 in some instances. In some instances, at least one of the one or more sensors, transducers, and/or other monitoring elements is positioned at the distal tip of the instrument 132.

Similar to instrument 130, instrument 132 also includes at least one element configured to monitor pressure within the vessel 100. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element are implemented as a solid-state component manufactured using semiconductor and/or other suitable manufacturing techniques. Millar catheters are utilized in some embodiments. Currently available catheter products suitable for use with one or more of Philips's Xper Flex Cardio Physiomonitoring System, GE's Mac-Lab XT and XTi hemodynamic recording systems, Siemens's AXIOM Sensis XP VC11, McKesson's Horizon Cardiology Hemo, and Mennen's Horizon XVu Hemodynamic Monitoring System and include pressure monitoring elements can be utilized for instrument 132 in some instances.

In accordance with aspects of the present disclosure, at least one of the instruments 130 and 132 is configured to monitor a pressure within the vessel 100 distal of the stenosis 108 and at least one of the instruments 130 and 132 is configured to monitor a pressure within the vessel proximal of the stenosis. In that regard, the instruments 130, 132 are sized and shaped to allow positioning of the at least one element configured to monitor pressure within the vessel 100 to be positioned proximal and/or distal of the stenosis 108 as necessary based on the configuration of the devices. In that regard, FIG. 3 illustrates a position 138 suitable for measuring pressure distal of the stenosis 108. In that regard, the position 138 is less than 5 cm, less than 3 cm, less than 2 cm, less than 1 cm, less than 5 mm, and/or less than 2.5 mm from the distal end of the stenosis 108 (as shown in FIG. 2) in some instances. FIG. 3 also illustrates a plurality of suitable positions for measuring pressure proximal of the stenosis 108. In that regard, positions 140, 142, 144, 146, and 148 each represent a position that is suitable for monitoring the pressure proximal of the stenosis in some instances. In that regard, the positions 140, 142, 144, 146, and 148 are positioned at varying distances from the proximal end of the stenosis 108 ranging from more than 20 cm down to about 5 mm or less. Generally, the proximal pressure measurement will be spaced from the proximal end of the stenosis. Accordingly, in some instances, the proximal pressure measurement is taken at a distance equal to or greater than an inner diameter of the lumen of the vessel from the proximal end of the stenosis. In the context of coronary artery pressure measurements, the proximal pressure measurement is generally taken at a position proximal of the stenosis and distal of the aorta, within a proximal portion of the vessel. However, in some particular instances of coronary artery pressure measurements, the proximal pressure measurement is taken from a location inside the aorta. In other instances, the proximal pressure measurement is taken at the root or ostium of the coronary artery.

In some embodiments, at least one of the instruments 130 and 132 is configured to monitor pressure within the vessel 100 while being moved through the lumen 106. In some instances, instrument 130 is configured to be moved through the lumen 106 and across the stenosis 108. In that regard, the instrument 130 is positioned distal of the stenosis 108 and moved proximally (i.e., pulled back) across the stenosis to a position proximal of the stenosis in some instances. In other instances, the instrument 130 is positioned proximal of the stenosis 108 and moved distally across the stenosis to a position distal of the stenosis. Movement of the instrument 130, either proximally or distally, is controlled manually by medical personnel (e.g., hand of a surgeon) in some embodiments. In other embodiments, movement of the instrument 130, either proximally or distally, is controlled automatically by a movement control device (e.g., a pullback device, such as the Trak Back® II Device available from Volcano Corporation). In that regard, the movement control device controls the movement of the instrument 130 at a selectable and known speed (e.g., 2.0 mm/s, 1.0 mm/s, 0.5 mm/s, 0.2 mm/s, etc.) in some instances. Movement of the instrument 130 through the vessel is continuous for each pullback or push through, in some instances. In other instances, the instrument 130 is moved step-wise through the vessel (i.e., repeatedly moved a fixed amount of distance and/or a fixed amount of time). Some aspects of the visual depictions discussed below are particularly suited for embodiments where at least one of the instruments 130 and 132 is moved through the lumen 106. Further, in some particular instances, aspects of the visual depictions discussed below are particularly suited for embodiments where a single instrument is moved through the lumen 106, with or without the presence of a second instrument.

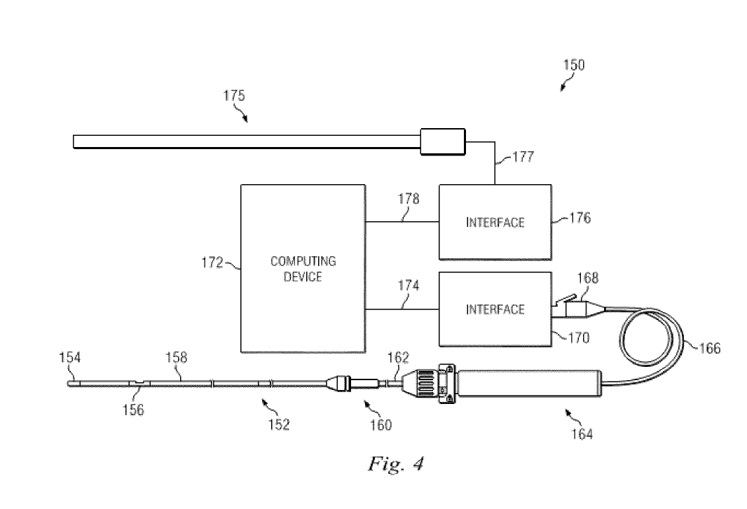

Referring now to FIG. 4, shown therein is a system 150 according to an embodiment of the present disclosure. In that regard, FIG. 4 is a diagrammatic, schematic view of the system 150. As shown, the system 150 includes an instrument 152. In that regard, in some instances instrument 152 is suitable for use as at least one of instruments 130 and 132 discussed above. Accordingly, in some instances the instrument 152 includes features similar to those discussed above with respect to instruments 130 and 132 in some instances. In the illustrated embodiment, the instrument 152 is a guide wire having a distal portion 154 and a housing 156 positioned adjacent the distal portion. In that regard, the housing 156 is spaced approximately 3 cm from a distal tip of the instrument 152. The housing 156 is configured to house one or more sensors, transducers, and/or other monitoring elements configured to obtain the diagnostic information about the vessel. In the illustrated embodiment, the housing 156 contains at least a pressure sensor configured to monitor a pressure within a lumen in which the instrument 152 is positioned. A shaft 158 extends proximally from the housing 156. A torque device 160 is positioned over and coupled to a proximal portion of the shaft 158. A proximal end portion 162 of the instrument 152 is coupled to a connector 164. A cable 166 extends from connector 164 to a connector 168. In some instances, connector 168 is configured to be plugged into an interface 170. In that regard, interface 170 is a patient interface module (PIM) in some instances. In some instances, the cable 166 is replaced with a wireless connection. In that regard, it is understood that various communication pathways between the instrument 152 and the interface 170 may be utilized, including physical connections (including electrical, optical, and/or fluid connections), wireless connections, and/or combinations thereof.

The interface 170 is communicatively coupled to a computing device 172 via a connection 174. Computing device 172 is generally representative of any device suitable for performing the processing and analysis techniques discussed within the present disclosure. In some embodiments, the computing device 172 includes a processor, random access memory, and a storage medium. The computing device 172 may also be connected to databases with medical information. In that regard, in some particular instances the computing device 172 is programmed to execute steps associated with the data acquisition and analysis described herein. Accordingly, it is understood that any steps related to data acquisition, data processing, instrument control, and/or other processing or control aspects of the present disclosure may be implemented by the computing device using corresponding instructions stored on or in a non-transitory computer readable medium accessible by the computing device. In some instances, the computing device 172 is a console device. In some particular instances, the computing device 172 is similar to the s5™ Imaging System or the s5i® Imaging System, each available from Volcano Corporation. In some instances, the computing device 172 is portable (e.g., handheld, on a rolling cart, etc.). Further, it is understood that in some instances the computing device 172 comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

Together, connector 164, cable 166, connector 168, interface 170, and connection 174 facilitate communication between the one or more sensors, transducers, and/or other monitoring elements of the instrument 152 and the computing device 172. However, this communication pathway is exemplary in nature and should not be considered limiting in any way. In that regard, it is understood that any communication pathway between the instrument 152 and the computing device 172 may be utilized, including physical connections (including electrical, optical, and/or fluid connections), wireless connections, and/or combinations thereof. In that regard, it is understood that the connection 174 is wireless in some instances. In some instances, the connection 174 includes a communication link over a network (e.g., intranet, internet, telecommunications network, and/or other network). In that regard, it is understood that the computing device 172 is positioned remote from an operating area where the instrument 152 is being used in some instances. Having the connection 174 include a connection over a network can facilitate communication between the instrument 152 and the remote computing device 172 regardless of whether the computing device is in an adjacent room, an adjacent building, or in a different state/country. Further, it is understood that the communication pathway between the instrument 152 and the computing device 172 is a secure connection in some instances. Further still, it is understood that, in some instances, the data communicated over one or more portions of the communication pathway between the instrument 152 and the computing device 172 is encrypted.

The system 150 also includes an instrument 175. In that regard, in some instances instrument 175 is suitable for use as at least one of instruments 130 and 132 discussed above. Accordingly, in some instances the instrument 175 includes features similar to those discussed above with respect to instruments 130 and 132 in some instances. In the illustrated embodiment, the instrument 175 is a catheter-type device. In that regard, the instrument 175 includes one or more sensors, transducers, and/or other monitoring elements adjacent a distal portion of the instrument configured to obtain the diagnostic information about the vessel. In the illustrated embodiment, the instrument 175 includes a pressure sensor configured to monitor a pressure within a lumen in which the instrument 175 is positioned. The instrument 175 is in communication with an interface 176 via connection 177. In some instances, interface 176 is a hemodynamic monitoring system or other control device, such as Siemens AXIOM Sensis, Mennen Horizon XVu, and Philips Xper IM Physiomonitoring 5. In one particular embodiment, instrument 175 is a pressure-sensing catheter that includes fluid column extending along its length. In such an embodiment, interface 176 includes a hemostasis valve fluidly coupled to the fluid column of the catheter, a manifold fluidly coupled to the hemostasis valve, and tubing extending between the components as necessary to fluidly couple the components. In that regard, the fluid column of the catheter is in fluid communication with a pressure sensor via the valve, manifold, and tubing. In some instances, the pressure sensor is part of interface 176. In other instances, the pressure sensor is a separate component positioned between the instrument 175 and the interface 176. The interface 176 is communicatively coupled to the computing device 172 via a connection 178.

Similar to the connections between instrument 152 and the computing device 172, interface 176 and connections 177 and 178 facilitate communication between the one or more sensors, transducers, and/or other monitoring elements of the instrument 175 and the computing device 172. However, this communication pathway is exemplary in nature and should not be considered limiting in any way. In that regard, it is understood that any communication pathway between the instrument 175 and the computing device 172 may be utilized, including physical connections (including electrical, optical, and/or fluid connections), wireless connections, and/or combinations thereof. In that regard, it is understood that the connection 178 is wireless in some instances. In some instances, the connection 178 includes a communication link over a network (e.g., intranet, internet, telecommunications network, and/or other network). In that regard, it is understood that the computing device 172 is positioned remote from an operating area where the instrument 175 is being used in some instances. Having the connection 178 include a connection over a network can facilitate communication between the instrument 175 and the remote computing device 172 regardless of whether the computing device is in an adjacent room, an adjacent building, or in a different state/country. Further, it is understood that the communication pathway between the instrument 175 and the computing device 172 is a secure connection in some instances. Further still, it is understood that, in some instances, the data communicated over one or more portions of the communication pathway between the instrument 175 and the computing device 172 is encrypted.

It is understood that one or more components of the system 150 are not included, are implemented in a different arrangement/order, and/or are replaced with an alternative device/mechanism in other embodiments of the present disclosure. For example, in some instances, the system 150 does not include interface 170 and/or interface 176. In such instances, the connector 168 (or other similar connector in communication with instrument 152 or instrument 175) may plug into a port associated with computing device 172. Alternatively, the instruments 152, 175 may communicate wirelessly with the computing device 172. Generally speaking, the communication pathway between either or both of the instruments 152, 175 and the computing device 172 may have no intermediate nodes (i.e., a direct connection), one intermediate node between the instrument and the computing device, or a plurality of intermediate nodes between the instrument and the computing device.

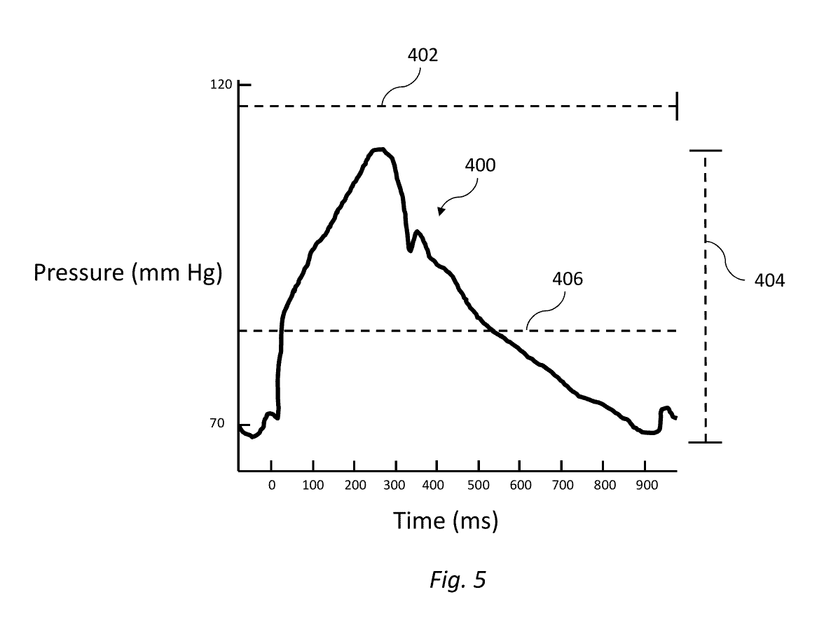

Referring now to FIGS. 5-7, shown therein are aspects of a technique for evaluating a vessel according to an embodiment of the present disclosure. In that regard, the technique described below with respect to FIGS. 5-7 may be implemented using any of the diagnostic measurements/calculations and associated techniques discussed above for evaluating a vessel across a lesion, stenosis, or region of interest. However, as will be discussed in greater detail, the technique associated with FIGS. 5-7 removes anomalous waveforms from a set of waveforms to yield more accurate results when analyzing cardiac data.

When making intravascular physiologic calculations, such as FFR, iFR, and CFR, the accuracy of the calculation can be adversely affected by anomalous cardiac waveforms. The ability to automatically detect such anomalous waveforms and remove (or process) the associated data from physiologic measurements/calculations in accordance with the present disclosure increases the accuracy of such measurements/calculations for diagnostic purposes. Below, automatic detection of anomalous cardiac waveforms is described along with filtering techniques for removing (or processing) the detected anomalous waveforms. In some implementations, a previously recorded set of waveforms (e.g., pressure, flow, and/or ECG waveforms) is analyzed to establish a baseline or reference waveform. For example, FIG. 5 illustrates a reference waveform 400 from a set of pressure measurements.

The reference waveform 400 can be defined by averaging or otherwise calculating one or more characteristics of a waveform set of physiologic data obtained for a particular patient. In this regard, it is understood that the waveform set utilized to establish the reference waveform 400 may be defined by any number of a waveforms, including 1, 2, 5, 10, 20, 50, 60, or more. For example, in some implementations the waveform set utilized to define the reference waveform is a collection of waveforms obtained over a certain number of cardiac cycles or amount of time. Further, in some implementations the waveform set is rolling or continually updated over time during a procedure such that the reference waveform can be updated in a corresponding manner. As an example, the waveform set may be defined by the last n heartbeat cycles, such that the reference waveform is continually updated based on the characteristics of the waveforms received over the last n heartbeat cycles. This approach can be particularly useful in situations where a known change in cardiac condition(s) is expected (e.g., application of a hyperemic drug) during a procedure. In other instances, the waveform set is fixed (e.g., during a setup period) and remains constant throughout a procedure such that the reference waveform 400 also remains fixed.

Further, the reference waveform may be utilized for a particular vessel, or even a particular portion of a vessel, such that multiple reference waveforms are provided for a patient. In this regard, it is understood that different vessels, and different portions of a single vessel, exhibit physiologic characterstics that may generate waveforms that would be considered anomalous for other vessels, or other portions of the same vessel, but should not be considered anomalous for that particular vessel or portion of the vessel. For example, ventricularization is particular severe in the right coronary artery. Accordingly, the reference waveform and/or the parameters utilized to compare subsequent waveforms to the reference waveform may be defined to can take this fact into consideration when determining whether the subsequent waveform is anomalous. Further, in some instances the reference waveform 400 is defined based on empirical data collected for a large population of patients. In this regard, the patient under examination may be classified into one or more groups of patients and a corresponding reference waveform selected based on the patient's classification(s). Accordingly, in such instances the reference waveform 400 is based on an expected waveform profile, instead of being based on actual waveform profiles of the patient. A database of reference waveforms can be maintained (and updated over time) such that medical personnel can choose the appropriate reference waveform(s) for a particular patient based on the patient's particular symptoms, anatomical location, medical history, etc.

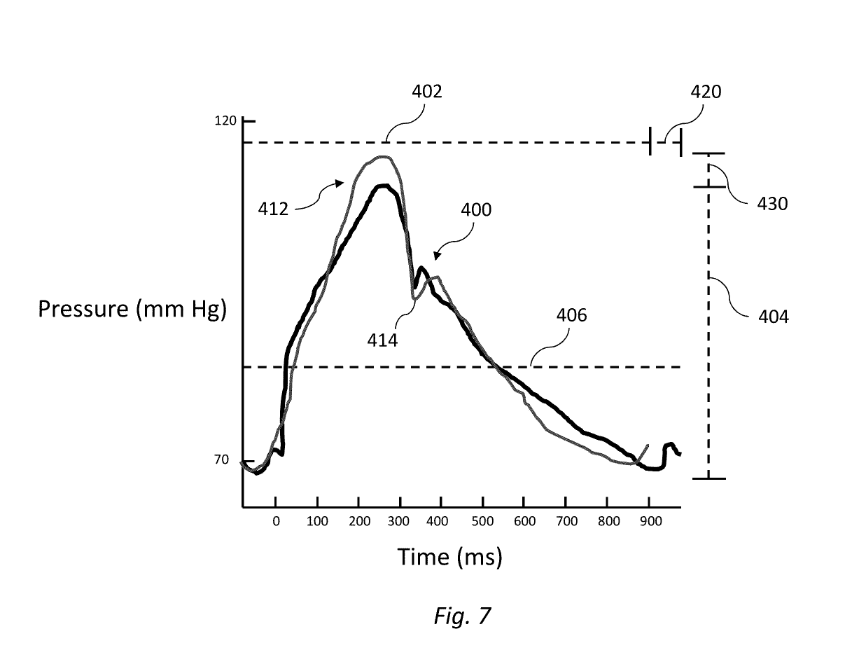

With the reference waveform 400 defined, subsequent waveforms, such as a patient waveform 412 (seen in FIG. 7), can be classified by discretizing it into a number of different characteristics and comparing those characteristics to the reference waveform 400. In this regard, the characteristics can include one or more of a minimum value, a maximum value, an average (mean) value, a median value, a range between minimum value and maximum value, a slope between two reference points (e.g., start, stop, maximum, minimum, dicrotic notch, etc.), a length (e.g., time) between two reference points (e.g., start, stop, maximum, minimum, dicrotic notch, etc.), presence or absence of a waveform characteristic (e.g., dicrotic notch, more than one systolic peak, a bump in a pressure wave near the end of the heartbeat cycle) and/or other measurable characteristic of the waveforms. In this regard, these characteristics can be evaluated using a wide variety of signal processing techniques, including transformations (e.g, Fourier transforms), regressions, inflections, derivatives, best fit analyses, etc. If the subsequent waveform does not correspond to the reference waveform 400 within a defined tolerance range (e.g., within x %, within y units of measurement (e.g., mmHg, seconds, etc.), etc.), then it can be considered anomalous and treated accordingly. The particular tolerance range can be set based on empirical data. In this regard, it is understood that by considering a large population of patients over time particular characteristics of a waveform may be found to have particular tolerance thresholds (e.g., very large or very small tolerances) that are suitable for particular physiologic measurement calculations. Therefore, the tolerance range can be defined accordingly for each characteristic. Further, in some instances the tolerance range may set at least partially based upon the particular physiologic measurement calculation(s) being made. As an example, iFR measurements that average pressure measurements within a wave-free period of a heartbeat cycle may allow for a higher variance in waveform characteristics outside of the wave-free period compared to FFR measurements that average over an entire heartbeat cycle.

If a subsequent waveform is determined to be anomalous, then the processing system can take this into account when making the physiologic measurement calculations. For example, in some instances all data from the anomalous waveform is simply excluded from the calculations. In other instances, at least some data from the anomalous waveform is included in the calculations. For example, in some instances some characteristics of a waveform may be within tolerance, while other characteristics are out of tolerance. In such instances, if the physiologic measurement calculation does not depend upon aspects of the waveform associated with the out of tolerance characteristic, then the data associated with the aspects of the waveform within tolerance may be used for making the physiologic measurement calculation. In some instances, data associated with aspects of the waveform that are out of tolerance are also utilized in the physiologic measurement calculations, but are conditioned to limit adverse effects on the calculations. For example, in some instances the data associated with the out of tolerance portion of a waveform is averaged with the data of corresponding sections of either a previous number of waveforms and/or the reference waveform such that the resulting average value is utilized. In other instances, the data associated with the out of tolerance portions are replaced with the running average of the last n waveforms and/or the reference waveform. In some implementations, a user is able to select how anomalous waveforms and/or associated data are treated for particular physiologic measurement calculations, including excluding all data from the anomalous waveform, excluding a portion of the data from the anomalous waveform, conditioning a portion of the data from the anomalous waveform, replacing a portion of the data from the anomalous waveform, and/or combinations thereof.

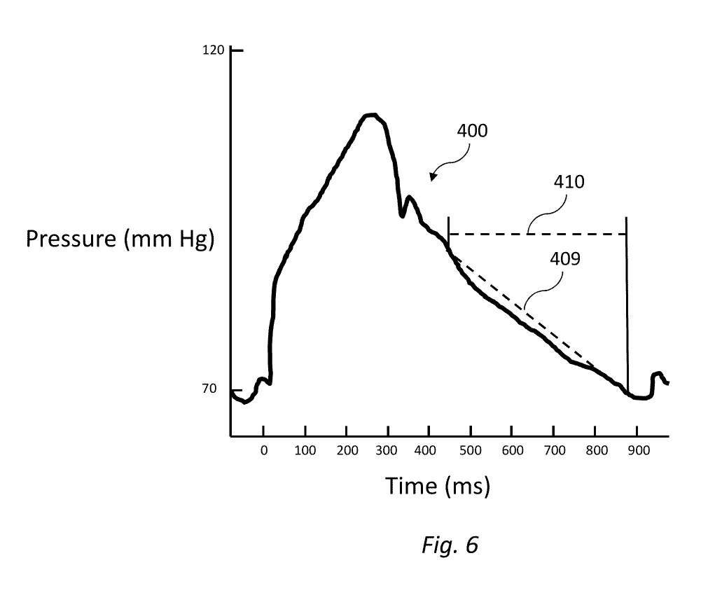

Generally, a sequence of patient waveforms are collected and analyzed to diagnose the health of a vessel. To increase the accuracy of calculations made based on the collected waveform set, each waveform 412 is compared to the reference waveform to identify anomalous waveforms. In some cases, medical professionals collect a set of waveforms for a particular patient at the beginning of a procedure to define the reference waveform 400. In doing so, medical professionals can select the amount and quality of data gathered and ensure that the reference waveform 400 will correspond closely to the patient's waveforms since it based on the patient's actual waveforms. The medical professionals may choose a previously recorded set of waveforms from the patient, from another patient, or from a reference set of waveforms defined for a group of patients that are similar in some respect to the patient, such as having similar ages, health conditions, instrumentation used in recording waveforms, etc. Creating a reference waveform 400 from a large set of recorded waveforms may also be favored to obtain a more accurate baseline. As noted above, the reference waveform 400 may be specific to a particular vessel of the patient and/or portion of a vessel. Accordingly, a plurality of reference waveforms may be defined for a single patient. Once a reference waveform 400 has been defined, each of the subsequent waveforms of the patient is compared to the reference waveform to identify anomalous waveforms. For example, key physical features of the waveforms are compared to detect significant variations. As shown in the exemplary embodiment of FIGS. 5 and 6, these physical features can include a waveform cycle length (time) 402, a mean pressure value 406, a range 404 of pressure values between a maximum pressure and a minimum pressure, and/or a slope 409 of pressure values obtained during a wave-free period 410, among various other waveform features.

Referring to FIG. 7, the patient waveform 412 is shown relative to the reference waveform 400 such that the waveforms can be compared. The total waveform cycle length 402 of the waveforms 400, 412 is compared. The difference 420 between the cycle lengths is determined and/or recorded by a computing device 172 and compared to an acceptable variation range to determine if the patient waveform 412 should be considered anomalous. The acceptable variation range may differ according to the procedure contemplated by the medical professional or the health of a patient. For example, if a medical professional depends heavily on the results the analysis alone in deciding whether or not to conduct a procedure, the acceptable variation range may be very small to produce a more accurate sample. If a medical professional instead is examining the overall health of the patient or trends in a patient's recovery, larger acceptable variation range may be used to create a more complete picture. In some embodiments, acceptable variation values for waveform cycle length 402 include variances greater than 50% of the total waveform cycle length 402 of the reference waveform 400. For example, in some instances a relatively large variation in heartbeat cycle length is caused by an ectopic heartbeat (e.g., resulting from premature ventricular contraction (PVC)). In particular, the ectopic heartbeat can result in a very short heartbeat followed by a very long heartbeat. In some implementations, ectopic heartbeat cycles are not considered anomalous and, therefore, are within the acceptable variation range. In some embodiments, acceptable variation values for waveform cycle length 402 are within 50% of the total waveform cycle length 402 of the reference waveform 400. In other embodiments, acceptable variation values are within 20% of the total waveform cycle length 402 of the reference waveform 400. In other embodiments, acceptable variation values are within 10% of the total waveform cycle length 402 of the reference waveform 400.

Similar to the waveform cycle length 402, the range of pressure values 404 between the two pressure values of the waveforms 400, 412 is measured. The range of pressure values may be measured between the highest and lowest pressure values as shown in FIG. 7, or alternatively at other points along the waveform, such as at the bottom of the dicrotic notch 414. The difference 430 between the range of pressure values 404 of the waveforms 400, 412 is then compared to an acceptable variation range to determine if the patient waveform 412 is anomalous. In some embodiments, acceptable variation for range of pressure values 404 are within 40% of the total pressure value 404 of the reference waveform 400. In other embodiments, acceptable variations are within 20% of the total pressure value 404 of the reference waveform 400. In other embodiments, acceptable variations are within 5% of the total pressure value 404 of the reference waveform 400.

The mean pressure values 406 of the waveforms 400, 412 may be compared and measured against an acceptable variation range to determine if the mean pressure value 406 of the patient's waveform 412 is anomalous. In some embodiments, acceptable variations for mean pressure value 406 are within 20% of the mean pressure value 406 of the reference waveform 400. In other embodiments, acceptable variations for mean pressure value 406 are within 10% of the mean pressure value 406 of the reference waveform 400. In other embodiments, acceptable variations for mean pressure value 406 are within 5% of the mean pressure value 406 of the reference waveform 400.

As shown in FIG. 6, the contiguous pressure values within the wave-free period (WFP) 410 of the waveform 400 may be analyzed. Because the resistance measured within a vessel is very low during the wave-free period, the pressure in the vessel steadily decreases. Thus, the pressure measured during the wave-free period of a waveform 400 generally trends downward and may assume a linear shape with a negative slope (e.g., slope 409). If the contiguous pressure values within the wave-free period 410 do not trend downward, it is unlikely that the waveform is accurate and should be excluded in most cases. In some embodiments, a linear regression is applied to the waveform 400 to approximate the slope 409 of the contiguous pressure values within the wave-free period 410. The slope 409 of the patient waveform 412 may be compared to the slope of the reference waveform 400 to determine whether the slope 409 is within tolerance or not. In some embodiments, acceptable variations for slope 409 are within 20% of the slope of the reference waveform 400. In other embodiments, acceptable variations for slope 409 are within 10% of the slope of the reference waveform 400. In other embodiments, acceptable variations for slope 409 are within 5% of the slope of the reference waveform 400.

Generally, pressure and/or ECG waveforms may be identified as anomalous when they exhibit irregular shapes. Common irregularities in waveform shape include waveform damping, lack of a dicrotic notch, abnormal spikes, and inverted waveforms. Damping is a common phenomenon among pressure waveforms, and may be observed by comparison of Pd pressure waveforms measured on the distal side of a stenosis (such as those measured with instrument 130 of FIG. 3) with Pa waveforms (such as those measured with instrument 132 of FIG. 3). Although the physical manifestations of damping may vary according to source, damping may cause a generalization of the waveform shape as well as lessening of the total pressure value 404 of the waveform. As in other examples, waveforms exhibiting substantial damping are to be identified and excluded from the waveform set (or otherwise treated). Comparing Pd/Pa pressure waveforms may also be helpful in excluding waveforms that do not have a visible dicrotic notch. FIG. 7 illustrates a dicrotic notch 414. The dicrotic notch 414 may be visible in only one of the waveforms, occurring more commonly in the Pa waveforms. Waveforms where no dicrotic notch is visible may be identified as anomalous and removed from the set. Anomalous waveforms may also exhibit abnormal spikes based on noise from an external source, or diagnostic phenomena such as guidewire whip or drift of pressure measurements obtained with a particular intravascular device. Likewise, inverted shapes such as inverted R-waves in ECG readings can provide a clear indication of an anomalous waveform. In some instances, an inverted waveform can be an indication that the equipment has been set up improperly (e.g., by switching wire connections). In some instances, a Pa waveform is compared to a Pa waveform obtained at the beginning of the procedure (e.g., during normalization) to identify anomalous Pa waveforms.

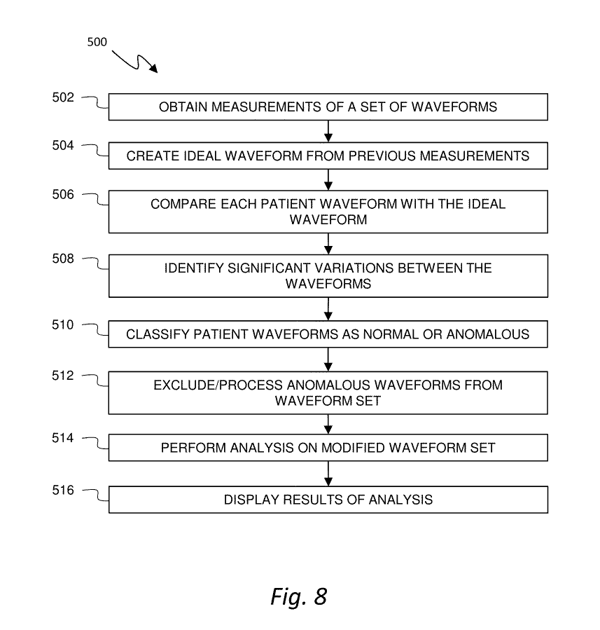

FIG. 8 is a flowchart illustrating a method 500 of automatically detecting anomalous waveforms and excluding them from use in making the physiologic measurements or processing them for use in making the physiologic measurements. For example, in some instances spikes in pressure values resulting from guide wire whipping can be filtered out of the waveforms. Similar filtering and/or processing techniques can be utilized to remove other types of anomalies in the waveforms. The method 500 will be described in the context of a pressure-sensing procedure, such as an iFR procedure, but may equally apply to any number of physiologic procedures, such as FFR, CFR, etc. The method 500 can be better understood with reference to the FIGS. 1-4.

Referring to FIG. 8, the method 500 begins at block 502 where one or more sets of waveform measurements are obtained using diagnostic instruments such as instruments 130, 132. The diagnostic instruments are also configured to obtain diagnostic information about the vessel 100. In one embodiment, these instruments 130, 132 are sized and shaped to allow positioning of at least one element configured to monitor pressure within the vessel 100 proximal of stenosis and at least one element configured to monitor pressure within the vessel 100 distal of stenosis 108. A variety of pressure sensors may be integrated with this instrument, such as piezo-resistive pressure sensors, piezo-electric pressure sensors, capacitive pressure sensors, electromagnetic pressure sensors, a fluid column, optical pressure sensors, and/or combinations thereof.

In block 504, a reference waveform is defined using previous waveform measurements. This reference waveform can be defined by averaging various characteristics of waveforms obtained during block 502. In other cases, a medical professional selects a reference waveform for the patient. This may involve selecting a reference waveform from a set of waveforms created by gathering and averaging sets of waveforms collected from other patients with similarities to the current patient, such as similar age, health conditions, measurements collected with similar instrumentation, etc. In some instances, the reference waveform(s) is automatically defined by the computing device 172 that collects the waveform data from instruments 130, 132.

At block 506, subsequent waveforms obtained by instruments 130, 132 are compared to the reference waveform. This step may be accomplished in several ways. A computing device 172 may measure a number of physical features of the reference waveform and assign numerical values to these features. The corresponding physical features of the subsequent patient waveforms are then compared to the reference physical features to determine whether the patient waveforms are within tolerance. Alternatively, the computing device 172 may graphically overlay the waveforms and determine if the patient's waveforms exceed a certain measurement range.

At block 508, a patient waveform having features outside of the accepted tolerance range are identified. For example, the computing device 172 can identify variations between the patient waveform and the reference waveform. As discussed above, these variations can include differences of waveform cycle length, different mean pressure values, different ranges of pressure values, differences in the contiguous pressure values within the wave-free period, damped waveforms, lack of a dicrotic notch, abnormal spikes, inverted waveforms, etc.

At block 510, the patient waveform is classified as either normal or anomalous. This classification can involves comparing the variations identified by the computing device with an acceptable variation for that feature to determine if the feature is anomalous. In some instances, the presence of a single anomalous features results in the patient waveform being characterized as anomalous. In other instances, the patient waveform is characterized as anomalous when a particular feature and/or combination of features are found to be anomalous. In other words, the patient waveform may be classified as normal even if one or more features are found to be anomalous. In this regard, it is understood that the acceptable variation values for the features of a waveform may vary according to the physiologic measurement calculations being made, user preferences, health of the patient, etc.

At block 512, the anomalous waveform is excluded from the patient waveform set or otherwise treated (e.g., filtered, scaled, processed, etc.) to limit the adverse effects of the anomalous feature(s) of the waveform on physiologic measurement calculations and, therefore, patient diagnosis. The exclusion or treatment of the anomalous waveform allows medical professionals to have higher confidence levels in the medical data and make better informed decisions concerning patient diagnosis and treatment.

At block 514, after the exclusion or treatment of the anomalous waveform(s), the computing device 172 performs an analysis and/or calculation using the modified waveform set in block 514. For example, in an iFR procedure, this analysis may include averaging the patient waveforms for the proximal and distal pressure measurements during a wave-free period and calculating a ratio of the distal pressure measurements to the proximal pressure measurements during the wave-free period.

At block 516, the results of the analysis and/or calculation are displayed to a user. These results may be in the form of numerical, graphical, textual, and/or other suitable visualizations, and may be displayed to medical professionals, patients, or caretakers and family members of patients.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method of evaluating a vessel of a patient, comprising:
collecting pressure data from an intravascular device positioned within the vessel of the patient, the pressure data including a pressure waveform for each cardiac cycle of the patient;
comparing the pressure waveform for each cardiac cycle of the patient to a reference pressure waveform to identify an anomalous pressure waveform; and
calculating a pressure ratio utilizing the pressure data from the intravascular device, wherein data from the anomalous pressure waveform is either excluded from the calculation or modified for use in the calculation.

2. The method of claim 1, wherein the reference pressure waveform is based on a previously recorded set of pressure waveforms of the patient.

3. The method of claim 2, wherein the reference pressure waveform remains fixed during a procedure.

4. The method of claim 2, wherein the reference pressure waveform varies during a procedure.

5. The method of claim 4, wherein the reference pressure waveform is based on n previous pressure waveforms obtained during the procedure.

6. The method of claim 1, wherein the reference pressure waveform is selected from a database of available pressure waveforms.

7. The method of claim 1, wherein comparing the pressure waveform for each cardiac cycle of the patient to the reference pressure waveform includes comparing a total cycle duration.

8. The method of claim 1, wherein comparing the pressure waveform for each cardiac cycle of the patient to the reference pressure waveform includes comparing at least one of a mean pressure, a range between a maximum pressure and a minimum pressure, or a slope of a portion of the waveform.

9. The method of claim 1, wherein comparing the pressure waveform for each cardiac cycle of the patient to the reference pressure waveform includes comparing a distal pressure waveform to a proximal pressure waveform.

10. The method of claim 1, further comprising identifying a group of anomalous waveforms based on comparing the pressure waveform for each cardiac cycle of the patient to the reference pressure waveform.

11. A system for evaluating a vessel of a patient, comprising:
at least one pressure-sensing intravascular device sized and shaped for positioning within the vessel of the patient; and
a processing system in communication with the at least one pressure-sensing device, the processing system configured to:
collect pressure data from the at least one pressure-sensing intravascular device, the pressure data including a pressure waveform for each cardiac cycle of the patient;
compare the pressure waveform for each cardiac cycle of the patient to a reference pressure waveform to identify an anomalous pressure waveform;
calculate a pressure ratio utilizing the pressure data from the intravascular device, wherein data from the anomalous pressure waveform is excluded from the calculation.

12. The system of claim 11, wherein the reference pressure waveform is based on a previously recorded set of pressure waveforms of the patient.

13. The system of claim 12, wherein the reference pressure waveform remains fixed during a procedure.

14. The system of claim 12, wherein the reference pressure waveform varies during a procedure.

15. The system of claim 14, wherein the reference pressure waveform is based on n previous pressure waveforms obtained during the procedure.

16. The system of claim 11, wherein the reference pressure waveform is selected from a database of available pressure waveforms.

17. The system of claim 11, wherein processing system is configured to compare the pressure waveform for each cardiac cycle of the patient to the reference pressure waveform by comparing a total cycle length.

18. The system of claim 11, wherein processing system is configured to compare the pressure waveform for each cardiac cycle of the patient to the reference pressure waveform by comparing at least one of a mean pressure, a range between a maximum pressure and a minimum pressure, or a slope of a portion of the waveform.

19. The system of claim 11, wherein processing system is configured to compare the pressure waveform for each cardiac cycle of the patient to the reference pressure waveform by comparing a distal pressure waveform to a proximal pressure waveform.

20. The system of claim 11, wherein processing system is further configured to identify a group of anomalous waveforms based on comparing the pressure waveform for each cardiac cycle of the patient to the reference pressure waveform.
